(19) United States
(12) Patent Application Publication
(10) Pub. No.: US 2019/0231316 A1
(43) Pub. Date: Aug. 1, 2019

(54) DIAGNOSIS AND MONITORING OF MYOCARDIAL INFARCTION USING ECG DATA FOR TREATMENT WITH SONOREPERFUSION ULTRASOUND

(71) Applicant: KONINKLIJKE PHILIPS N.V., Eindhoven (NL)

(72) Inventors: Jonathan SUTTON, Boston, MA (US); Richard GREGG, Westford, MA (US); Patrick RAFTER, Windham, NH (US); Jeffry POWERS, Bainbridge Island, WA (US)

(21) Appl. No.: 16/258,754

(22) Filed: Jan. 28, 2019

Related U.S. Application Data
(60) Provisional application No. 62/624,886, filed on Feb. 1, 2018.

(51) Int. Cl. A61B 8/08; A61B 8/00; A61B 5/044

(52) U.S. Cl. CPC A61B 8/483; A61B 8/463; A61B 8/06; A61B 8/481; A61B 8/4488; A61B 5/044

(57) ABSTRACT

An ECG system acquires ECG signals for the production of ECG lead data which is analyzed for ST elevation characteristics. The ST elevation information is further analyzed to identify a coronary artery or region of the myocardium which may be affected by an obstruction or occlusion. The identity of an obstructed coronary artery or affected myocardium is related to a spatial location in an ultrasound image of the heart to target an ultrasound probe at the coronary artery or myocardial region which may be obstructed or affected by an obstruction. Sonoreperfusion is then performed at the coronary artery or myocardial region targeted by the ultrasound probe.

This application is the U.S. application which claims the benefit of U.S. patent application Ser. No. 62/624,886, filed on Feb. 1, 2018. This application is hereby incorporated by reference herein.

This invention relates to medical diagnostic and therapy systems and, in particular, to a system which targets coronary artery blood clots and occlusions using ECG lead data and treats identified obstructions and affected tissue with sonoreperfusion ultrasound.

A heart attack, or myocardial infarction, occurs when a coronary artery supplying blood to a portion of the myocardium becomes obstructed by plaque or a blood clot. When a subject is suspected of having suffered a myocardial infarction, generally the first test performed is an electrocardiogram (ECG) exam by which the ECG lead waveforms are analyzed to determine whether the episode was a “STEMI” heart attack or an “NSTEMI” heart attack. A STEMI episode, which stands for ST-elevation myocardial infarction, is often a complete blockage of a coronary artery. An NSTEMI, or non-ST-elevation myocardial infarction, is often a partial blockage or high-grade blockage but with collateral circulation. The determination of the type of episode dictates various types of treatment which may be performed. A partial blockage may be treated with clot-dissolving drugs or balloon angioplasty. A more serious complete blockage may be treated by cardiac catheterization or coronary artery bypass grafting. For treatments physically applied at the site of the obstruction such as angioplasty, it is necessary to locate the site in the body. A conventional way to do this is by taking an angiogram of the patient's heart after injecting radiographic dye into the right or left coronary arteries from the aorta, which reveals the pathways of blood vessels and their obstructions. With an obstruction targeted from visualization of the coronary pathways, a revascularization treatment such as angioplasty can be applied to the obstructed artery.

These procedures are invasive and time-consuming, as they must be done carefully and properly. An angiogram exposes the patient to the radiographic contrast agent and the risk of dissection or perforation of a coronary artery. Revascularization procedures must be performed by skilled trained surgeons. If a coronary obstruction could be located quickly and easily, such as with an initial electrocardiogram, the information could be used to perform a targeted therapy. It has recently been determined that, by analysis of the characteristics of the waveforms of an electrocardiogram, the portion of the coronary artery network which is obstructed can be identified. See, for instance, U.S. Pat. No. 8,233,971 (Zhou et al.) and U.S. Pat. No. 9,462,955 (Zhou et al.) As explained in these patents, a diagnostic ECG exam can be performed to generally locate the culprit coronary artery which has suffered an obstruction. It would be desirable to be able to use the results of an ECG exam such as described in these patents to be able to identify and directly treat at-risk tissue.

In accordance with the principles of the present invention, a diagnostic ECG procedure is used to locate an obstructed coronary artery and diagnostic and therapeutic ultrasound is then used to visualize the site and apply sonoreperfusion to break up the obstruction and effect vasodilation in an ischemic cardiac volume. While the result of the diagnostic ECG may not exactly pinpoint the obstructed branch of the arterial network, its spatial precision is generally sufficient for the ultrasound treatment, as a flow of microbubbles can reach throughout the coronary network and the applied ultrasound can insonify a wide area of an ischemic cardiac volume to effect sonoreperfusion clot breakup and vasodilation. A combined system is described which is able to provide both the ECG localization and the ultrasonic therapeutic effect.

In the drawings:

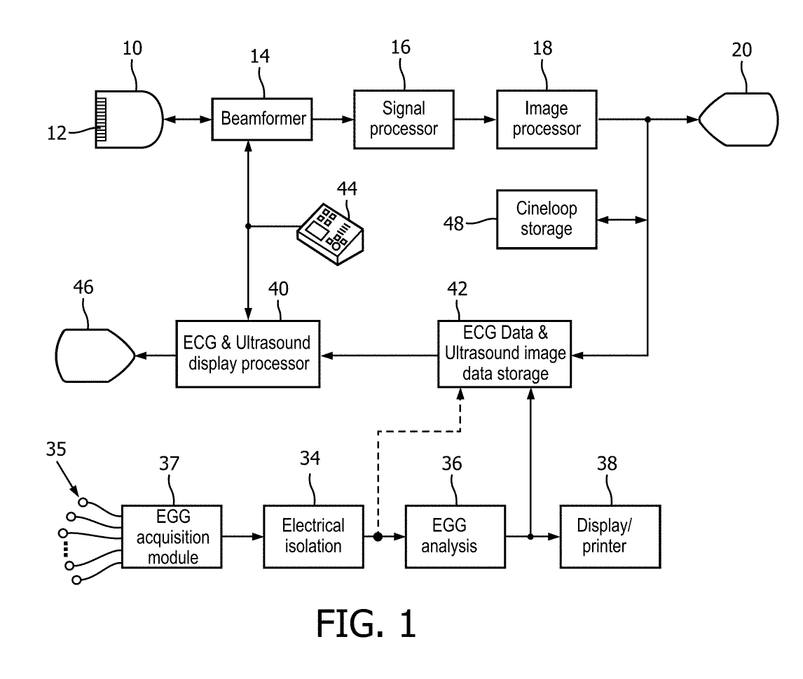

FIG. 1 illustrates in block diagram form an ultrasound and ECG diagnostic system constructed in accordance with the principles of the present invention.

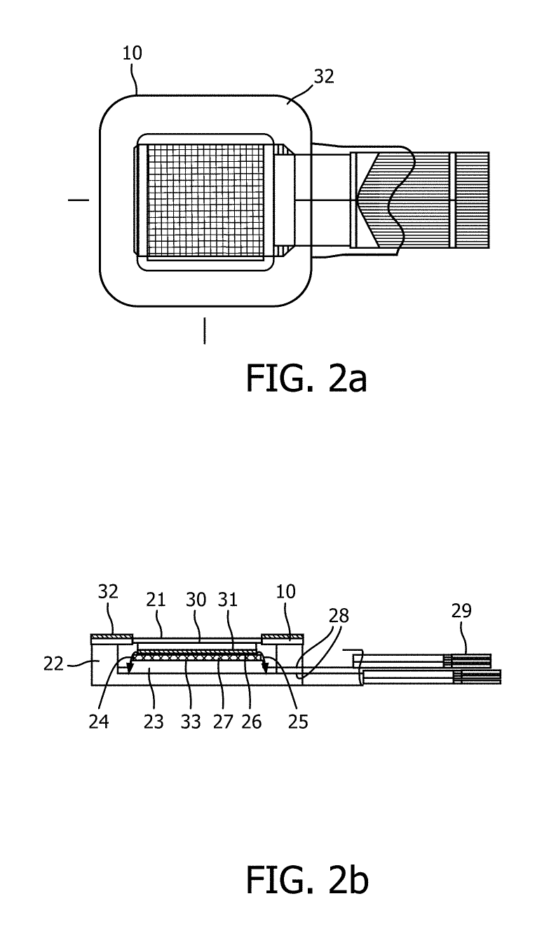

FIGS. 2a and 2b illustrate an ultrasonic matrix array probe which can be adhesively attached to the body for imaging and therapy.

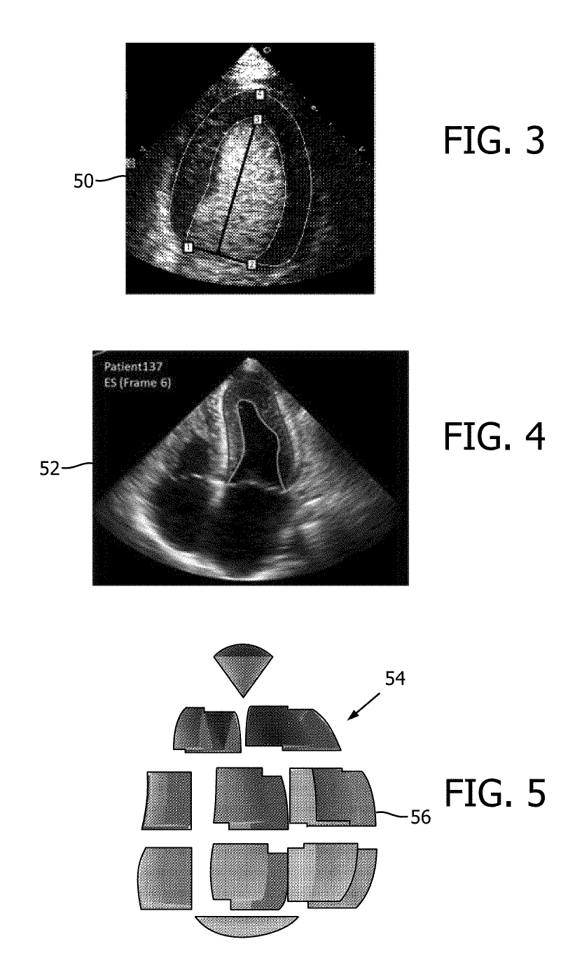

FIGS. 3 and 4 illustrate ultrasound views of the heart in which the myocardium has been identified and traced using a heart model.

FIG. 5 is a flowchart explaining the operation of a typical heart model algorithm.

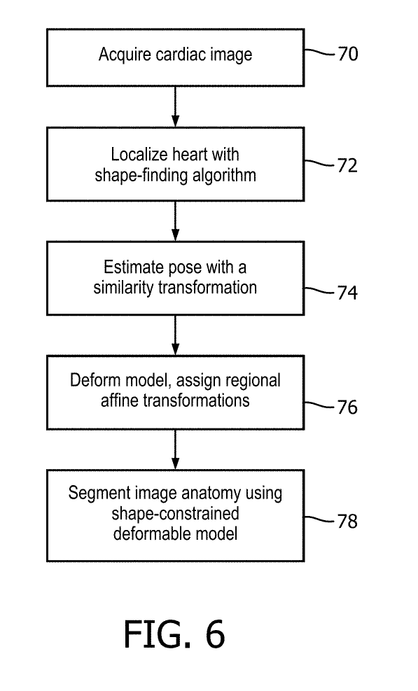

FIG. 6 is an exploded view of a 3D heart model.

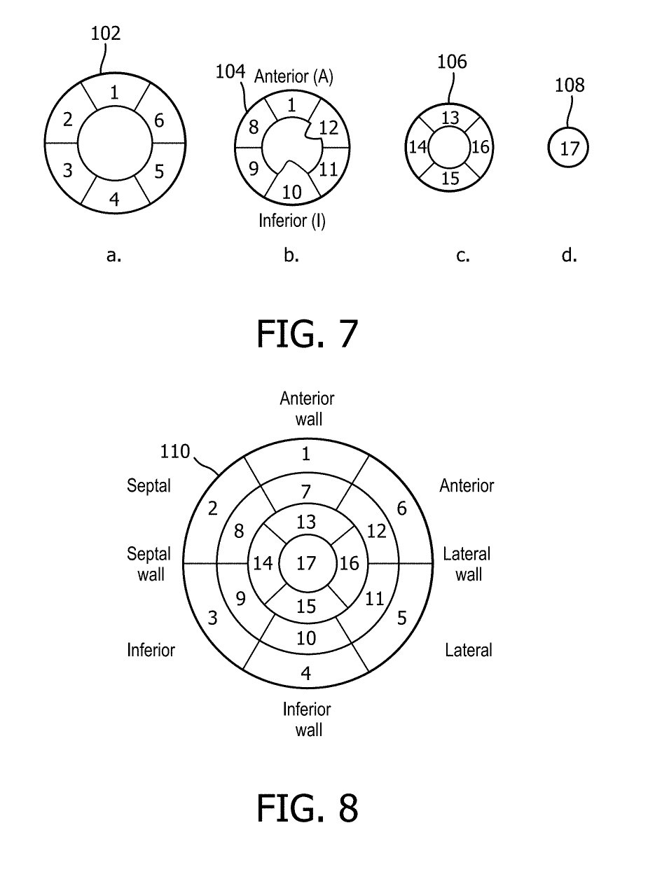

FIGS. 7 and 8 illustrate the layout of a bullseye chart on which 3D ultrasound segmentation and ECG diagnostic information can be displayed in a combined anatomical presentation.

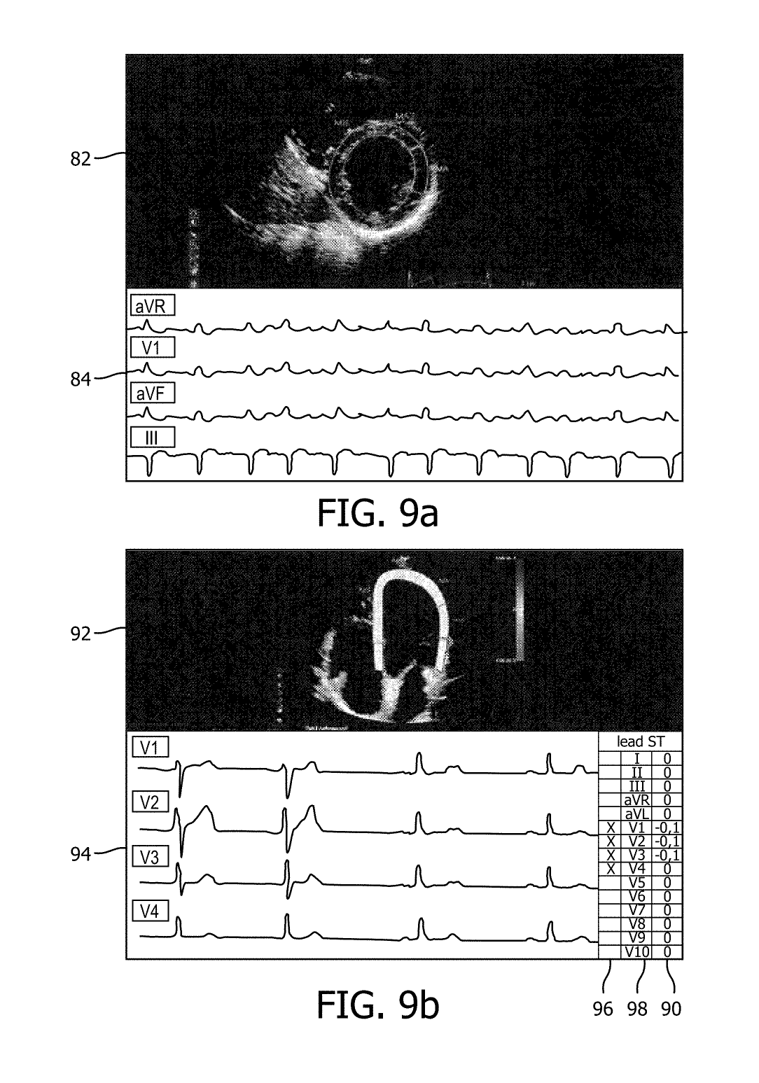

FIG. 9a illustrates a short axis ultrasound view of the heart and associated ECG lead traces on the same display.

FIG. 9b illustrates a combined ultrasound image display and ECG trace display with user selection of the specific lead traces to be shown for the particular ultrasound image view.

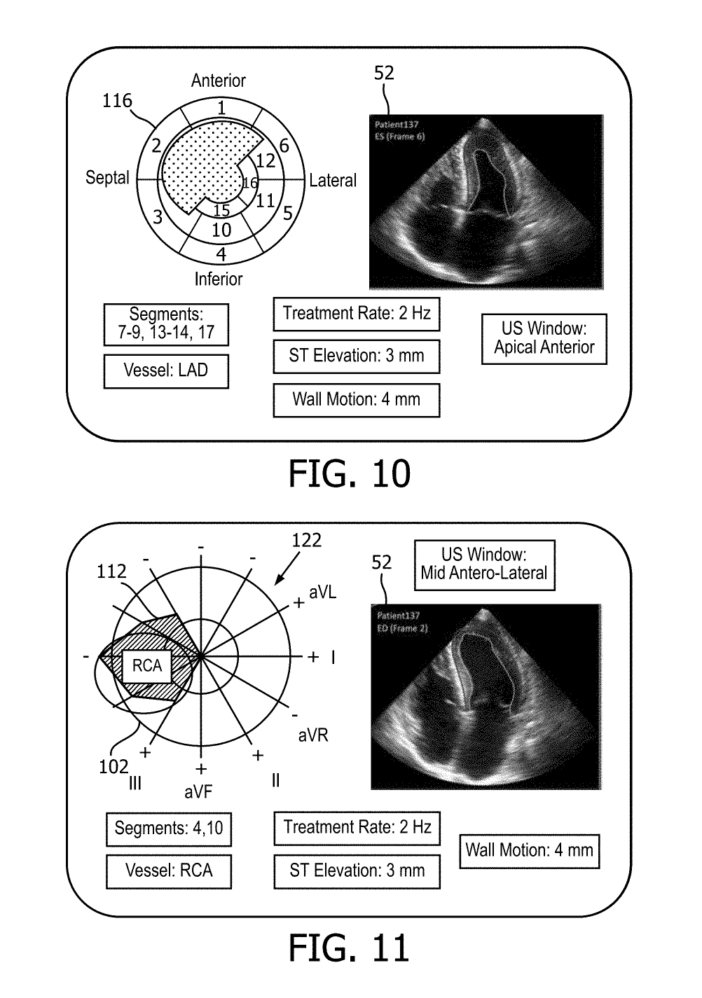

FIG. 10 illustrates a screen display of diagnostic ECG information on a bullseye chart, an ultrasound image, and sonothrombolysis treatment information for an ECG and ultrasound system constructed in accordance with the present invention.

FIG. 11 illustrates a screen display of diagnostic ECG information on a spidergram chart, an ultrasound image, and sonothrombolysis treatment information for an ECG and ultrasound system constructed in accordance with the present invention.

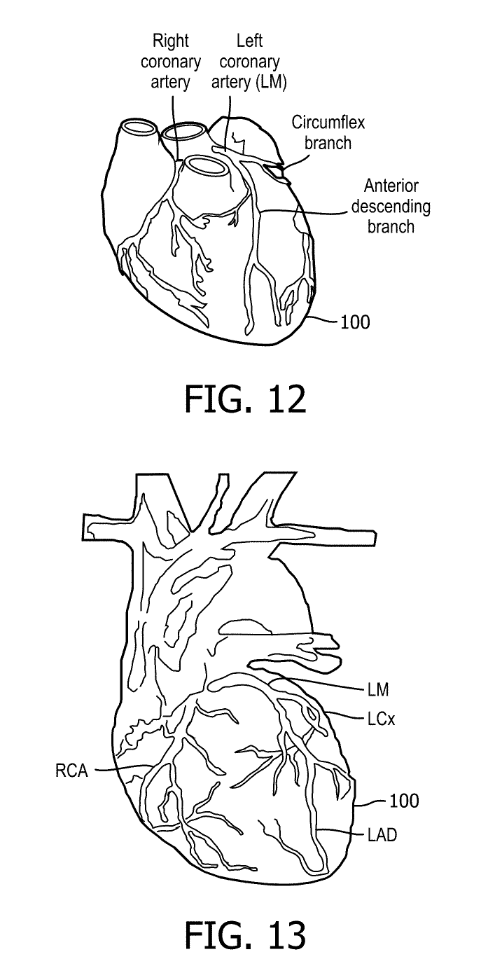

FIGS. 12 and 13 are diagrams of a heart showing the locations of the major coronary arteries which may be targeted and treated by sonothrombolysis in accordance with the present invention.

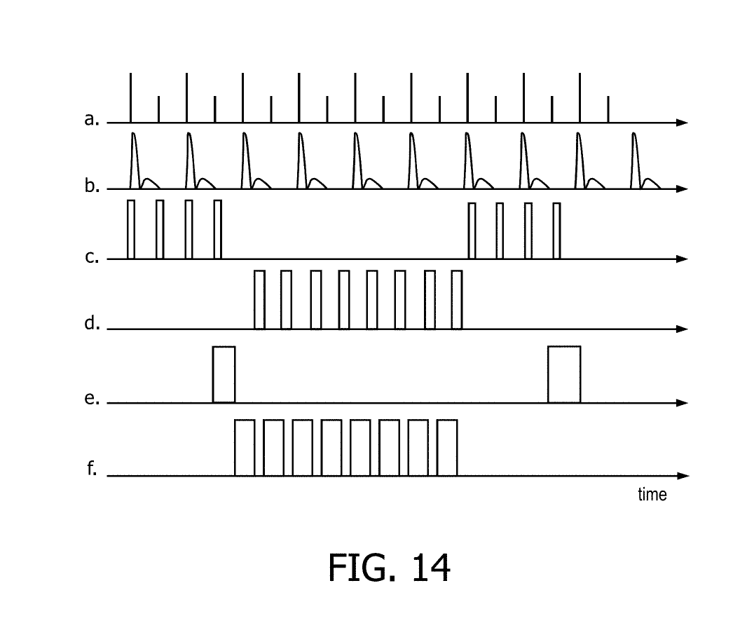

FIG. 14 is a timing diagram of a typical sonothrombolysis treatment regimen.

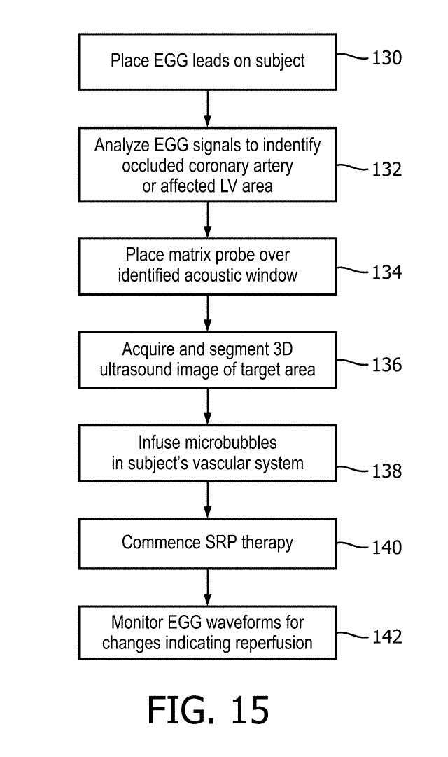

FIG. 15 is a flowchart of a diagnostic ECG and sonothrombolysis diagnosis and treatment procedure for a coronary artery obstruction in accordance with the present invention.

Referring first to FIG. 1, a combined diagnostic ECG and ultrasound imaging and sonothrombolysis system is shown in block diagram form. The major subsystems of the ultrasound portion of the system are shown at the top of the drawing. An ultrasound probe 10 with an array transducer 12 transmits ultrasound waves or pulses to the heart of a patient under control of a beamformer 14 and, during imaging, receives echoes in response. The echo signals received by the individual transducer elements of the array are processed by delaying and summing them in the beamformer 14 to form coherent echo signals relating to specific points in the body. The echo signals are processed by a signal processor 16. Signal processing may include separation of harmonic echo signal components for harmonic imaging and clutter removal, for example. The processed signals are arranged into images of a desired format by an image processor 18. When only a plane of the body is scanned by the probe, a 2D image is produced, and when a 3D volume within the body is scanned, three dimensionally arranged echo signals are received and processed to produce 3D images. The image processor in this implementation also includes an image border detector, preferably a heart model processor, which will be described below. The images produced by the image processor can be displayed on an ultrasound system display 20. Live image loops are stored in Cineloop® memory 48 for later recall and analysis.

The major subsystems of the diagnostic ECG portion of the system are shown at the bottom of the drawing. Electrodes 35 are attached to the skin of the patient at specific locations on the body to acquire ECG signals. Usually the electrodes are disposable conductors with a conductive adhesive gel surface that sticks to the skin. Each conductor has a snap or clip that snaps or clips onto an electrode wire of the ECG system. A typical ECG system will have twelve leads (ten electrodes), which may be expanded with additional leads on the back of the patient and the right side of the chest for up to sixteen leads. Extended lead sets with up to eighteen leads may be used. In addition, fewer leads such as 3-lead (EASI and other), 5-, and 8-lead sets can also be used to derive 12 leads, but with reduced accuracy. The acquired ECG signals, which are on the order of millivolts, are preconditioned by an ECG acquisition module 37 which performs processing such as amplification, filtering and digitizing of the ECG signals. The electrode signals are coupled to an ECG analysis module 36, generally by means of an electrical isolation arrangement 34 that protects the patient from shock hazards and also protects the ECG system if the patient is undergoing defibrillation, for instance. Optical isolators are generally used for electrical isolation. The ECG analysis module combines the signals from the electrodes in various ways to form the desired lead signals, and performs other functions such as signal averaging, heart rate identification, and identifies signal characteristics such as the QRS complex, the P-wave, T-wave, and other characteristics such as elevation seen in the ST segment of a waveform. The processed ECG information is then displayed on an image display or printed in an ECG report by an output device 38.

In accordance with the principles of the present invention, the ultrasound images and the ECG lead data are coupled to a combined ultrasound and ECG system. In FIG. 1 the ultrasound and ECG information are coupled to an ECG data and ultrasound image data storage device 42. In the configuration of FIG. 1 the ultrasound and ECG functionality are resident on a single system. Other arrangements are also possible such as a stand-alone ultrasound system connected to a stand-alone cardiograph. In the system of FIG. 1, data from the two functionalities are directly coupled to an ECG data and ultrasound image data storage device 42. Alternatively, the data may be coupled to the device 42 over a network, or may be ported into the device 42 on one or a plurality of media storage devices. The ECG data and ultrasound image data are then processed for common display by an ECG and ultrasound display processor 40. The combined data is then displayed on an image display 46, which may be of a form described in FIGS. 10 and 11 below. A control panel 44 is operated by a user to control the processing and display of the merged data and also to control the sonothrombolysis treatment as explained below. In other implementations, the storage device 42, the processor 40, the control panel 44 and the display 46 are a workstation or a separate computer system.

FIGS. 2a and 2b illustrate a preferred ultrasound probe 10 for an implementation of the present invention, which is a two-dimensional matrix array of transducer elements. The matrix ultrasound transducer is formed as a patch that adheres to the patient's body with double-sided medical grade tape 32 so that it can remain in the same position during sonothrombolysis treatment of a specific coronary artery. A suitable matrix array patch is described in U.S. Pat. No. 6,685,647 (Savord et al.), which uses a de-matching layer for a low-profile assembly. The matrix array is formed as a standard piezoelectric based acoustic stack connected through a ball grid or equivalent interconnect to a microbeamformer ASIC behind the array. FIG. 2a shows a top view of the matrix array probe 10. FIG. 2b shows the probe in a sectional view illustrating the construction of the matrix array stack. As seen in FIG. 2b there is an acoustic window 21; acoustic matching layers 30; piezoelectric elements 31; the adhesive tape 32; a plastic housing 22; a microbeamforming ASIC 25; an acoustic de-matching layer 26; a stud bump or ball grid array in conductive epoxy used to connect the array elements to the microbeamforming ASIC 27 and therefore provides conductivity between the two; an epoxy backfill 33 that isolates the individual conductive elements from each other; a flexible circuit 23 coupled to the ASIC; a wire band ASIC-to-flexible-circuit interconnect 24; flexible circuits 28 to couple the probe to the ultrasound system by means of a coax cable array 29. In use, the central matrix array area of the probe (see FIG. 2a) is and acoustically coupled to a patient's body in the area of interest with ultrasonic gel.

The matrix array probe of FIGS. 2a and 2b is particularly useful in an implementation of the present invention, where the probe is often attached to the chest of the patient to access the heart parasternally for imaging or therapy. Generally, an array transducer transmits beams which are steered over a sector-shaped (or pyramid-shaped) field of view, which has its apex at the center of the array. But it may happen that the center of the array is blocked from accessing the heart by rib-shadowing. With a matrix array, the apex of the scan field can be translated toward a side of the array, where it is no longer blocked from accessing the heart by rib-shadowing.

As mentioned above, the ultrasound image processor 18 includes an image border detector. The purpose of the border detector is to automatically identify and trace the borders of the myocardium so that specific regions of the myocardium can be segmented and identified. The result of border detection of the myocardium of the left ventricle (LV) in an ultrasound cardiac image is shown in FIG. 3. This ultrasound image is a contrast-enhanced harmonic image in which the chamber of the LV has been flooded with a contrast agent but the agent has not yet fully perfused the myocardium, which is why the LV chamber appears very bright against the darker surrounding myocardium in this image. When a user clicks on the outer myocardial apex at point 5 in the image, an automated border detection (ABD) processor selects an outer or epicardial template of the LV epicardium and fits it to the outside of the myocardium, fitting it to previously identified mitral valve corners 1 and 2, as illustrated in FIG. 3. A similar process initially fits the mitral valve corners and the endocardial apex 3 to an endocardial shape. The cardiac image now has both its endocardial boundary, the blood pool-myocardium interface, and its epicardial boundary, the interface between the trabeculated myocardium and the compacted myocardium, delineated in the image by tracings as shown in

FIG. 3. Automatic border detection processors are described in U.S. Pat. No. 6,491,636 (Chenal et al.), U.S. patent publication no. 2005/0075567 and PCT publication no. 2005/054898, for instance. Automatic border detection is useful in an implementation of the present invention because the coronary arteries are located on the epicardium.

FIG. 4 illustrates an ultrasound image with both myocardial boundaries traced at end systole. The epicardial boundary is traced with a darker graphic line and the endocardial boundary is traced with a lighter graphic line in this image. These myocardial boundaries have been traced by a preferred technique for automatically delineating the myocardial borders is with a deformable heart model. A heart model resident in the image processor 18 contains shapes and/or meshes of selected regions of the heart and cardiovascular system, such as the atria, ventricles, epicardial boundary and endocardial border shapes of the chambers of the heart. See U.S. Pat. No. 7,010,164 (Weese et al.) and “Automatic Model-Based Segmentation of the Heart in CT Images” by Ecabert et al., published in IEEE Trans. On Med. Imaging, vol. 27, no. 9 (Sept. 2008) at pp 1189-1201. A heart model is a spatially-defined mathematical description of the tissue structure of a typical heart which can be fitted to the heart as it appears in a diagnostic image, thereby defining the specific anatomy of the imaged heart. Unlike a standard heart model designed to identify interior structures of the heart such as valves and chambers, a heart model of the present invention operates to locate multiple myocardial boundaries, including both an inner endocardial boundary and the outer epicardial boundary. The processing performed by a preferred heart model is illustrated in FIG. 5. The process begins with the acquisition of a cardiac image at 70. The position of the heart is then localized in the cardiac image by processing the image data with a shape-finding algorithm such as a generalized Hough transform at 72. At this point the pose of the heart has not been defined, so misalignments in translation, rotation and scaling of the heart in the image data are corrected by use of a single similarity transformation for the whole heart model at 74. Next at 76, the model is deformed and affine transformations are assigned to specific regions of the heart. Constraints on the deformation are then relaxed by allowing the heart model to deform with respect to the piecewise affine transformation at 78, and the shape-constrained deformable model is resized and deformed so that each part of the model fits the actual patient anatomy as shown in the image at the captured phase of the heart cycle (step 70), including both an inner and outer myocardial boundaries. The model is thus accurately adapted to the organ boundaries shown in the cardiac image, thereby defining the boundaries including the endocardial lining and the epicardial boundary. In a preferred implementation of such a heart model, the epicardial boundary is found first, as this typically appears as a well-defined gradient between a brightly illuminated region and a region of moderate illumination in an ultrasound image. The endocardial boundary is generally less well-defined in a heart model due to the desire to be able to find the variable location of the less well-defined endothelial lining as it appears in an ultrasound image. Unlike the contrast-enhanced cardiac image of FIG. 3, an unenhanced ultrasound image such as that of FIG. 4 will generally exhibit a relatively sharp intensity gradient between the relatively high intensity echo tissue surrounding the myocardium and the medium intensity of the myocardium, and a relatively lesser gradient between the myocardium and the low intensity of the chamber's blood pool. This mandates in favor of discriminating the outer myocardial border first, then the inner endocardial boundary when diagnosing images acquired in the absence of a contrast agent. When the coordinates of a boundary have been found, they are communicated to a graphics generator of the image processor, which generates the traces that overlie the displayed ultrasound image in the calculated positions.

If the heart model of a three-dimensional image of a heart myocardium were perfectly egg-shaped, it could be segmented into eighteen discrete segments as illustrated by the segments 56 of the three-dimensional heart model 54 of FIG. 6. Seventeen of these segments (all but the top segment) are of anatomical segments of the heart which can be numbered and are locationally specific to a cardiologist; a cardiologist would know from a segment number exactly which portion of the myocardium is indicated. Thus, a 3D ultrasound image of the heart can have its myocardium traced by a heart model to delineate the endocardium and the epicardium of the myocardium in the ultrasound heart image, then segmented by the heart model into seventeen locationally specific segments so that a clinician can refer to a specific segment in a report if an abnormality in a region of the myocardium is diagnosed. If a region of the heart has suffered an infarction, for example, the clinician may diagnose an akinetic condition at a certain segment and so indicate on the diagnostic report.

In accordance with the principles of the present invention, the seventeen locationally-specific heart segments are mapped to a two-dimensional bullseye chart as shown in FIGS. 7 and 8. In these illustrations the segments of a bullseye chart have been numbered in correspondence with the anatomy of the heart in a standardized pattern as shown in the drawings. Myocardial segments of a basal short axis ultrasound view 102, near the mitral valve plane, are numbered 1 through 6 as shown in FIG. 7a. The smaller circle 104 of FIG. 7b represents the segments of a mid-cavity short axis view, with the segments numbered 7 through 12. The lower apical level short axis view 106 of FIG. 7c has four segments numbered 13 through 16. Each of these three ultrasound image plane circles is oriented to the anterior side of the heart at the top, to the inferior side of the heart at the bottom, to the septal wall to the left and to the lateral wall of the heart at the right. A final segment 17 is added for the apex of the heart as shown at 108 in FIG. 7d. These circles are displayed concentrically as a bullseye chart 110 as shown in FIG. 8. The concentric bullseye is three dimensional in nature, as it is anatomically oriented around the chart to the four sides of the heart, and from the outer diameter to the center in accordance with different levels of the heart.

In a first implementation of the present invention a bullseye chart 110 is annotated with information produced by the diagnostic ECG subsystem that identifies the location of an ischemic tissue. This information is developed from ST-elevation data of the ECG leads as illustrated in FIGS. 9a and 9b. FIG. 9a shows a display screen with a short axis, mid-cavity view of the heart in an ultrasound image in display area 82. The border of the myocardium has been traced and segmented over the heart myocardium. Since the short axis view shows a complete myocardial path around the heart, anterior, lateral, inferior and septal segments of the myocardium are seen in the ultrasound image. There are a number of ECG leads which anatomically correspond to this view and its segments, including leads aVR, V1 and V2 for the anteroseptal segments, leads aVL, I, V5 and V6 for the anterolateral segments, leads aVF, III, V1 and V2 for the inferoseptal segments, and leads II, aVF, V5 and V6 for the inferolateral segments. In this example leads corresponding to the septal region of the myocardium are displayed in the ECG lead display area 84, which are the aVR, V1, aVR, and III lead signals.

FIG. 9b shows an apical 4-chamber view of the heart in the ultrasound display area 92, but in this example a clinician has selected a different set of leads for concurrent display with this view. As seen in ECG lead display area 94, the clinician has selected leads V1, V2, V3, and V4 for display with this ultrasound view. At the right side of the ECG lead display area are three columns of ECG lead information. The middle column 98 shows all of the ECG leads of the lead set used for the diagnosis. In the left column 96 the user has entered “Xs” next to the leads which are to be displayed in the display area 94. As this example illustrates, the user has selected leads V1, V2, V3 and V4 for viewing. Since the display area can display four lead traces at the illustrated level of resolution, the user can place Xs next to any four leads, and the traces for the four selected ECG leads are shown in the display area 94. The column 90 to the right of the ECG lead column 98 is annotated with the value of ST elevation detected at each lead. In this example the negative values indicate that ST depression has been detected at leads V1, V2, and V3, and so the user has chosen to display the traces for leads V1-V4. The user can save the lead selections corresponding to particular views, such as V1-V4 for the apical 4-chamber view of FIG. 6, and can recall the selections and/or alter them by relocating the Xs in column 96 of the display.

In accordance with the first implementation of the present invention, a bullseye chart is produced with annotations of ECG ST-elevation values, thus providing an anatomical guide to the location of the region of ischemia/infarction. The user can view the annotated bullseye chart alone, but preferably it is concurrently viewed with an ultrasound image as shown in FIG. 10 so the clinician can see the correlation between the locational information of the bullseye chart and the spatial anatomical information of the ultrasound image. While the bullseye chart can be filled in with numerical ST-elevation data if desired, in the display screen example of FIG. 10 the bullseye chart is annotated with color shading of the myocardial segments where ST elevation has been identified. In bullseye chart 116 ST elevation is indicated at segments 13 and 14 and surrounding segments from leads V1-V2 to V3-V6, extending over the apical segment 17. This chart tells the clinician that therapy should be directed at the coronary artery network at the apical-anterior region of the heart, and myocardial tissue in this region. This identification of the culprit coronary artery can be seen from the anatomical illustrations of the coronary arteries shown in FIGS. 12 and 13.

In accordance with a further aspect of the present invention, this identification of the culprit coronary artery in the bullseye chart 116 is used to guide sonothrombolysis treatment of the coronary artery occlusion. The display screen illustration of FIG. 10 is seen to identify the myocardial segments affected by the possible ischemia/infarction, segments 7-9, 13-14, and 17. Also identified on the display screen is the possible culprit coronary artery, the LAD. Below the ultrasound image window is an identification of the ultrasound window for viewing the suspect region, which is an apical anterior window. For viewing and therapy the array probe is attached to the body to view the heart from this window, which is in an apical anterior view from below the ribcage. When treatment commences as discussed below, significant treatment parameters are displayed in the center of the display screen as shown.

FIG. 11 shows a display screen of a second implementation of the present invention which uses a spidergram-type of chart for the ST-elevation information instead of a bullseye chart. This type of display is described in detail in international patent publication number WO 2006/033038 (Costa Ribalta at al.) which is incorporated herein by reference. The spidergram display technique presents ECG data in a way that allows rapid detection of data in its spatial orientation. Two and three dimensional graphical illustrations are presented in this patent publication. These graphical displays give information not only about the current values of ST segment data but also about the spatial arrangement of the data. Thus, an annotated spidergram display presents an anatomically-oriented graphic of ECG data from which a clinician can quickly identify a culprit coronary artery which is obstructed and a possible cause of an acute ischemic event.

Two different spidergram orientations are possible, one for the ECG limb lead signals and another for the ECG chest lead signals. The upper left area of the display screen of FIG. 11 shows a spidergram graphic 122 of limb lead signals, which are approximately in a vertical plane of an erect subject. The limb leads are depicted at various positions around a circle in relation to their view of the heart. These leads include the aVR, aVL and aVF leads shown in the graphic 122, as well as the I, II, and III leads which are also developed from limb electrode signals. The graphic includes axes for the signals which are oriented in relation to the limb positions of an erect subject with arms extended outward, with the axis for the I lead being the horizontal (0°) axis in the drawing and the II and III lead axes disposed on opposite sides of the vertical (90°) aVF axis. The ends of the axes are scaled in mm of ST elevation, the millimeter notation being familiar to most cardiologists. The translation from the electrical units measured by the Lou system to the millimeter notation is 100 microvolts equals 2 millimeters. The axes in the graphic 122 are also seen to have + and − polarities. A lead exhibiting an ST elevation will have the data value plotted on the positive side of the axis from the origin, and ST depression measurements are plotted on the remaining negative side of the axis. The ST elevation values for the limb electrodes are plotted on the respective axes of the graphic and the points plotted on the lead axes are connected by lines with the area inside the lined shape 112 colored or shaded as shown in the drawing. In this example, a sizeable shape 112 is formed in the limb lead graphic 122 by the significant ST elevation values measured for leads II, III, and aVF, and the ST depression values measured for leads I and aVL.

A similar graphic can be provided for the chest leads. The axes for the chest leads are in a horizontal plane and are arrayed from V1 through V6 in the same order as they are physically oriented on the chest. Axes for the chest leads V7-V9 which continue around the torso to the back of the chest can also be included to further fill out the array of axes in a chest lead spidergram graphic. The chest lead graphic uses + and − polarities for the ST elevation and depression values in the same manner as the limb lead graphic. The ST elevation and depression values are similarly plotted as points on the respective lead axes and the points connected to form a shape in the same manner as the limb lead graphic 122.

The locations of the ECG-derived shapes in the anatomically related spidergram graphics are used to visually identify suspect culprit coronary arteries. In the limb lead graphic 122 an ECG-derived shape which is located in the upper right region of the graphic is generally symptomatic of obstruction of the left anterior descending (LAD) coronary artery. A shape located around the left center of the graphic is usually indicative of a right coronary artery (RCA) obstruction. Obstruction of the left circumflex coronary artery (LCx) is signaled by a shape located around the bottom center of the graphic. The locations of ECG-derived shapes signaling possible LCx, RCA, and LAD obstruction in the chest lead graphic are similarly located in the upper portion, the left portion, and the lower right portion of the graphic, respectively. Thus, a clinician can take a quick look at the spidergram graphic display and immediately see which coronary artery is the probable cause of an ischemic condition. The large shape 112 in the lower left quadrant of the limb lead graphic 122 of FIG. 11 would suggest obstruction of the right coronary artery (RCA).

Like the display of FIG. 10, the display screen of FIG. 11 identifies myocardial segments where the infarction is probably located, segments 4 and 10 in this example, as well as the identity of the RCA as the culprit coronary artery. The ultrasound window for probe placement is located above the ultrasound image, here shown as a mid antero-lateral view. This example, as well as the previous FIG. 10 example, shows the value of an ultrasonic measurement of wall motion, which is also an indicator of ischemia/infarction. As before, parameters of sonothrombolysis treatment are presented in the center of the screen.

FIGS. 12 and 13 are anatomical views of the locations of the coronary arteries on the front and back of the heart. FIG. 12 shows the right coronary artery (RCA) descending along the right side of the heart 100 from the aorta. Also descending from the aorta along the left side of the heart is the left main (LM) coronary artery, which quickly branches to form the left anterior descending (LAD) artery on the front (anterior) of the heart and the left circumflex (LCx) artery which wraps around the back (posterior) of the heart. In FIG. 13 the heart 100 is depicted as a translucent orb so that the tortuous paths of the coronary arteries on both the anterior and posterior sides of the heart can be readily visualized. All three major vessels are seen to ultimately wrap around the heart 100 in characteristic tortuous paths to provide a constant supply of fresh blood to the myocardium. These illustrations are presented to illustrate the relation between the identity of the suspected obstructed coronary artery and the ultrasound windows used to image and treat them by sonothrombolysis.

FIG. 14 is a timing diagram illustrating the conduct of a typical sonothrombolysis procedure designed to break up a blood clot in a coronary artery. Line a. shows tall and short lines representing the times of systolic and diastolic cardiac activity. Line b. shows ECG waveforms acquired during the heartbeats of line a. The systolic and diastolic activity is shown to align with the QRS and T wave respectively for clarity of illustration, but there is a significant delay between the electrical activity and the mechanical contraction as a response to the electrical activity. After the target region of the heart has been identified as described above, the matrix array probe is attached to the body so as to view the target anatomy from the indicated acoustic window. As shown in line c., 3D (volumetric) images of the heart are acquired and segmented (line e.) to update the aiming so as to assure that the proper region of the heart is being insonified. As shown in line d., color Doppler and contrast images may also be acquired for analysis, interleaved with the updating B mode 3D images.

Once the target anatomy is being imaged and insonified, a microbubble contrast agent is infused into the subject's bloodstream and allowed to flow to the coronary arteries. After arrival of the contrast agent in the coronary arteries has been ascertained by visualization in the ultrasound image, one or more beams are transmitted to the target artery each heart cycle to agitate or break up the microbubbles, activity which will lyse a blood clot and vasodilate small arterioles. See, e.g., U.S. Pat. No. 8,012,092 (Powers et al.) and U.S. Pat. No. 8,211,023 (Swan et al.) for further details on sonothrombolytic treatment. While sonothrombolysis and sonoreperfusion can be performed at elevated ultrasound intensity levels, sonothrombolysis and sonoreperfusion can often be effectively performed at mechanical index and thermal ultrasonic effect levels that are within the limits of standard diagnostic ultrasound. This enables sonoreperfusion to be performed by ultrasound systems used for standard imaging procedures.

FIG. 15 summarizes how the ultrasound and ECG system implementations described above may be used to diagnose and treat myocardial ischemia and infarction. In step 130 ECG electrodes are placed on a subject. The ECG leads acquire ECG signals, which are analyzed in step 132 to identify a coronary artery which may be obstructed by a blood clot or an area of the left ventricle which has been affected by ischemia. With the target coronary artery or myocardial area identified, a matrix array probe is attached to the patient in step 134 so as to image the target anatomy through the proper acoustic window. A 3D image of the heart is acquired and segmented in step 136 to assure that the probe is aimed at the desired anatomy for sonoreperfusion treatment. In step 138 microbubbles are infused into the subject's vascular system and thereafter sonoreperfusion treatment commences as indicated by step 140. The effects of the treatment are monitored during the therapy in step 142 for reperfusion improvement, as by monitoring for decreases in ST elevation values and/or improved cardiac wall motion, factors indicating reduction of an occlusion. Other diagnostic techniques can be used to monitor and direct the therapy, such as wall motion analysis, perfusion imaging and Doppler imaging. See, e.g., U.S. Pat. No. 7,043,062 (Gerard et al.) and U.S. Pat. No. 6,692,438 (Skyba et al.)

It should be noted that an ultrasound system and an ECG system suitable for use in an implementation of the present invention, and in particular the component structure of the combined ultrasound and ECG system of FIG. 1, may be implemented in hardware, software or a combination thereof. The various embodiments and/or components of such systems, for example, the signal processor and image processor of the ultrasound subsystem and the ECG acquisition and analysis modules and the display processor or components and controllers therein, also may be implemented as part of one or more computers or microprocessors. The computer or processor may include a computing device, an input device, a display unit and an interface, for example, for accessing the Internet. The computer or processor may include a microprocessor. The microprocessor may be connected to a communication bus, for example, to access a PACS system or the data network for importing training images. The computer or processor may also include a memory. The memory devices such as the Cineloop storage device and the ECG data and ultrasound image data storage device of FIG. 1 may include Random Access Memory (RAM) and Read Only Memory (ROM). The computer or processor further may include a storage device, which may be a hard disk drive or a removable storage drive such as a floppy disk drive, optical disk drive, solid-state thumb drive, and the like. The storage device may also be other similar means for loading computer programs or other instructions into the computer or processor.

As used herein, the term “computer” or “module” or “processor” or “workstation” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of these terms.

The computer or processor executes a set of instructions that are stored in one or more storage elements, in order to process input data. The storage elements may also store data or other information as desired or needed. The storage element may be in the form of an information source or a physical memory element within a processing machine.

The set of instructions of an ultrasound system or a diagnostic ECG system including those controlling the acquisition, processing, and display of ultrasound images or ECG signals as described above may include various commands that instruct a computer or processor as a processing machine to perform specific operations such as the methods and processes of the various embodiments of the invention. The set of instructions may be in the form of a software program. The software may be in various forms such as system software or application software and which may be embodied as a tangible and non-transitory computer readable medium. Further, the software may be in the form of a collection of separate programs or modules such as a neural network model module, a program module within a larger program or a portion of a program module. The software also may include modular programming in the form of object-oriented programming. The processing of input data by the processing machine may be in response to operator commands, or in response to results of previous processing, or in response to a request made by another processing machine.

Furthermore, the limitations of the following claims are not written in means-plus-function format and are not intended to be interpreted based on 35 U.S.C. 112, sixth paragraph, unless and until such claim limitations expressly use the phrase “means for” followed by a statement of function devoid of further structure.

What is claimed is:

1. A method for diagnosing and treating coronary artery disease comprising:
acquiring ECG signals from a subject;
analyzing the ECG signals to identify an ischemic cardiac volume for treatment;
directing an ultrasound probe toward the ischemic cardiac volume to image the anatomy; and
performing sonoreperfusion at the ischemic cardiac volume.

2. The method of claim 1, further comprising infusing the subject with a microbubble contrast agent.

3. The method of claim 1 wherein analyzing the ECG signals further comprises forming ECG lead waveforms from the acquired ECG signals.

4. The method of claim 3, wherein analyzing the ECG signals further comprises detecting ST elevation or depression in the ECG lead waveforms.

5. The method of claim 4, wherein analyzing the ECG signals further comprises identifying at-risk tissue which may be affected by an obstruction using the detected ST elevation or depression.

6. The method of claim 5, wherein identifying at risk tissue further comprises graphically identifying a coronary artery which may be obstructed.

7. The method of claim 6, wherein graphically identifying a coronary artery which may be obstructed further comprises displaying ST elevation or depression graphically.

8. The method of claim 7, wherein displaying ST elevation or depression graphically further comprises displaying ST elevation or depression in a bullseye chart.

9. The method of claim 7, wherein displaying ST elevation or depression graphically further comprises displaying ST elevation or depression in a spidergram graphic.

10. The method of claim 1, wherein directing an ultrasound probe toward at-risk tissue further comprises performing 3D ultrasound imaging of the heart.

11. The method of claim 10, wherein directing an ultrasound probe toward at-risk tissue further comprises segmenting a 3D ultrasound image of the heart.

12. The method of claim 11, wherein directing an ultrasound probe toward at-risk tissue further comprises relating a segment of a 3D ultrasound image to an identified coronary artery.

13. The method of claim 1, wherein directing an ultrasound probe toward at-risk tissue further comprises positioning an ultrasound probe to view the heart from a specific acoustic window.

14. The method of claim 13, wherein directing an ultrasound probe toward at-risk tissue further comprises affixing the ultrasound probe to the subject to view the heart from a specific acoustic window.

15. The method of claim 1, further comprising displaying to the user an identification of a coronary artery which may be obstructed and a region of a myocardium which may be obstructed.
